(19) United States
(12) Patent Application Publication
(10) Pub. No.: US 2026/0308512 A1
(43) Pub. Date: Oct. 8, 2026

(54) APPARATUS

(71) Applicant: Molecular Devices (UK) Limited, Wokingham (GB)

(72) Inventors: Jessica Pinheiro De Lucena-Thomas, Wokingham (GB); Marianne Ellis, Wokingham (GB); Kimberley Luetchford, Wokingham (GB)

(21) Appl. No.: 19/482,227

(22) PCT Filed: May 8, 2024
(86) PCT No.: PCT/GB2024/051191

§ 371 Date: Nov. 6, 2025

(30) Foreign Application Priority Data
May 9, 2023 (GB) 2306791.1

(51) Int. Cl. C12N 5/00; C12M 1/00; C12M 1/04; C12M 1/12; C12M 1/34

(52) U.S. Cl. CPC C12N 5/0062; C12M 23/24; C12M 23/40; C12M 25/20; C12M 29/12; C12M 41/40; C12M 47/04; C12N 5/0012; C12N 2533/50; C12N 2533/70; C12N 2533/74

(57) ABSTRACT

A method of expanding organoids includes: i) dissociating organoids into single cells and encapsulating the cells in gel beads; ii) culturing the encapsulated cells in an incubator under static conditions; iii) transferring the encapsulated cells to a fluidised bed bioreactor of a cell expansion apparatus, with a pump providing about 0.1 mm/s to about 5 mm/s superficial flow velocity in the bioreactor, to form organoids; and iv) retrieving the gel beads from the fluidised bed bioreactor for harvesting of the organoids. A cell expansion apparatus is also disclosed.

### CROSS-REFERENCE TO RELATED APPLICATION(S)

This application is the national phase of PCT Application No. PCT/GB2024/051191 filed on May 8, 2024, which in turn claims priority to British Application No. 2306791.1 filed on May 9, 2023, the contents of which are incorporated herein by reference in their entireties.

### STATEMENT REGARDING FEDERALLY SPONSORED RESEARCH OR DEVELOPMENT

The project leading to this application has received funding from the European Union's Horizon 2020 research and innovation programme under the Marie Skkodowska-Curie grant agreement No 665992.

### FIELD

The present disclosure relates to methods and cell expansion apparatus for culturing organoids.

### BACKGROUND

Prior to testing new drug candidates in vivo on animals or humans, in vitro testing is usually carried using either primary cell cultures or cell lines. However, results from this testing can be unreliable as the cell cultures do not mimic an in vivo system very well. This can lead to some good drugs being rejected at the in vitro stages and some poor drugs may be progressed to in vivo trials.

Organoids are three-dimensional structures of heterogeneous tissue that function like an in vivo tissue. In other words, these three-dimensional structures of tissue mimic an organ better than a traditional cell culture monolayer. Organoids therefore provide an opportunity to create cellular models of disease, which can be studied to better understand the causes of disease and identify possible treatments.

Organoids are often generated from stem cells, which can be differentiated into cerebral, renal, cardiovascular and other types of organoids. Organoid technology has also been used to create a model of human colon cancer progression. These organoids may be created from normal intestinal cells mutated to transform into cancer cells, or may be derived from tumour cells per se. Organoids created from tumours have been shown to be a good reflection of the original tumour, providing opportunities for improved in vitro drug testing. By delivering improved predictive capacity, the use of patient-derived organoids has the potential to revolutionize drug discovery, reducing costs and time commonly associated with late failures in clinical trials, with greater patient benefit and reduced animal usage.

Organoids have been increasingly used in research settings, however, the high potential of organoids for industrial applications has not yet been fully realised. Several different aspects play a role in the formation of organoids and the currently used manual and static organoid cultures are not a suitable approach to translate this technology from basic research to streamlined manufacturing.

Indeed, organoid culture is still in its early stages and is largely inefficient. Organoids are traditionally encapsulated into 10-50 μL adherent droplets of Matrigel® and cultured under static conditions in well-plates or dishes with nutrients given in semi-batch (Fatehullah et al., 2016). It is a labour intensive and costly process that produces small outputs with great variability between batches. Moreover, traditional cell culture in well plates, dishes and flasks is notoriously wasteful in terms of media components and single-use lab consumables. To make organoid culture suitable for mainstream adoption into the drug discovery process there is significant need to optimise the organoid production to a streamlined, efficient, and reliable process.

### SUMMARY

In static cell cultures, mass transfer (i.e., exchange of nutrients and gases) is limited by pronounced concentration gradients due to the build-up of inhibitory waste products and depletion of nutrients. Under static conditions, nutrient supply and waste removal happens solely by diffusion, and is thus limited to distances around 200 μm, which is approximately the distance that transfer of oxygen becomes limited by diffusion in tissues. Alternatively, in dynamic cultures using bioreactors, cells are within more homogeneous environments due to mixing, providing cells with improved mass transfer, which can simulate biological, physical and mechanical environments, as well as encouraging formation of complex structures.

Some dynamic systems include spinner flasks, rocking bioreactors and wave bioreactors, but they do not mimic well the in vivo microenvironment in the body. Better alternatives for organoid culture are perfusion bioreactors, which can offer downward, cross flow or microgravity environments. The latter including rotating wall and fluidised bed bioreactors, which offers greater mass transfer.

Bioreactors have been used to culture organoids in only a few instances, such as those summarized in Table 1. Some of these studies have suggested that using bioreactors not only substantially improves the generation of a variety of 3D suspension organoids when compared with conventional static organoid culture, but also increases the differentiation yield for those derived from pluripotent stem cells (PSCs) (Ovando-Roche et al., 2018). Indeed, organoid generation from PSCs (embryonic or induced), such as brain and retina organoids, has been boosted by bioreactors. These bioreactors improved mass transfer and allowed for extended culture times and larger organoid sizes, which is not possible in static conditions due to concentration gradients and lack of vasculature in organoids. For instance, bioreactors have been used to enhance nutrient absorption by brain organoids, allowing them to reach around 4 mm diameter and be cultured for nearly 10 months (Lancaster et al., 2013). Stirred tanks were the majority of bioreactors used, and rather than for large-scale production, they were mainly used for mass transfer purposes to improve organoid maintenance over extended periods to obtain larger organoid sizes (rather than organoid quantity), which is difficult in static conditions due to the lack of vasculature in organoids, the lack of vasculature contributing to the formation of concentration gradients and in worse cases, necrotic cores.

TABLE 1
Culture systems used for organoid culture

| | | Encapsulation | Bioreactor | | |
|---|---|---|---|---|---|
| | | method and | size and | | |
| Culture | Organoid | matrix | operating | Other remarks and | |
| system | type | composition | conditions | applications | Reference |
| Perfusion | Colorectal | Organoids seeded | Fed-plate flat- | For manufacturing of larger | (Ellis et al., |
| bioreactors | and breast | from single cells in | bed bioreactor | organoid batches. | 2018) |
| (milifluidic | cancer | a layer of Matrigel ® | based on | Improved reproducibility | |
| systems) | organoids | placed on the | 100 mm | and batch consistency for | |
| | (hASC- | bottom of the | culture dishes. | commercialisation. | |
| | derived) | bioreactor dish. | | Reduced personal and | |
| | | | | processing time costs. | |
| | | | | Fairly scalable. | |
| | Brain | 3D colonies | 2 mL modules | For organoid maintenance | (Berger et al., |
| | organoids | generated from an | with one | and protocol optimization | 2018) |
| | (hiPSC- | ultra-low | droplet per | (e.g. multiple parallel | |
| | derived) | attachment plate | chamber | operating conditions) | |
| | | and then seeded | operated at | Millifluidic system | |
| | | into droplets of | 240 μLmin⁻¹. | commercially available | |
| | | LDEV-free and | | (Quasi-Vivo ®) | |
| | | growth factor | | | |
| | | reduced GelTrex | | | |
| | | matrix. | | | |
| Stirred tank | Brain | Cell aggregates | 125 mL stirred | Organoids were maintained | (Lancaster |
| bioreactor | organoids | embedded into | tank with 75- | for up to 15 months, | and Knoblich, |
| | (hPSC- | 30 μL droplets of | 100 mL culture | however they stopped | 2014a) |
| | derived) | Matrigel ® (one per | media, which | growing after 2 months | |
| | | droplet), left 4 | was changed | and after 6-7 months, they | |
| | | days at stationary | once per week. | gradually shrank in size, | |
| | | culture and then | | likely due to progressive | |
| | | transferred to the | | loss of progenitor cells. | |
| | | bioreactor. | | Not more than 32 | |
| | | | | organoids in the | |
| | | | | bioreactor, as too many | |
| | | | | droplets in the bioreactor | |
| | | | | would lead to organoids | |
| | | | | fusing together. | |
| | Retinal | N/A | 100 mL stirred | Quicker development of | (Ovando- |
| | organoids | | tank at 22 rpm | photoreceptors using | Roche et al., |
| | (hPSC- | | and media | bioreactors (week 11) than | 2018) |
| | derived) | | changed once | in static plates (week 15). | |
| | | | per week | | |
| | Kidney | N/A | 125 mL stirred | Organoids developed | (Przepiorski et |
| | organoids | | tank, 90 rpm, | tubular structures by day | al., 2018) |
| | (hiPSC- | | half volume of | 8, and in vivo-like tissue | |
| | derived) | | media changed | morphology at day 14. Not | |
| | | | every 2 days | all the embryonic bodies | |
| | | | | formed organoids, with | |
| | | | | smaller aggregates often | |
| | | | | failing to generate tubular | |
| | | | | tissue. | |
| | Liver | Cells seeding of | 125-mL | Reached 40-fold cell | (Schneeberger |
| | organoids | 10⁵ cells · ml⁻¹ of | spinner flasks, | expansion after 2 weeks, | et al., 2019) |
| | (hASC- | expansion medium | 85 rpm | compared with 6-fold | |
| | derived) | supplemented with | | expansion in static | |
| | | 10% (v/v) | | cultures. | |
| | | Matrigel ® cultured | | | |
| | | the cells for two | | | |
| | | weeks and | | | |
| | | compared the | | | |
| | | organoids | | | |
| | | conventionally | | | |
| | | cultured in | | | |
| | | Matrigel ® droplets | | | |
| | | (static controls; | | | |
| | | 10⁵ cells/100 μl | | | |
| | | Matrigel ®). | | | |
| | Small | Organoids | 100 mL stirred | The core created a | (Lu et al., |
| | intestinal | encapsulated | tank, 6 days of | permissive environment for | 2017) |
| | organoids | within core-shell | culture | organoid growth while the | |
| | (Murine | capsule of around | | shell enabled the formation | |
| | ASC- | 1.4 mm diameter. | | of mechanically robust | |
| | derived) | The core was made | | capsules. | |
| | | of Matrigel ® and | | Expansion efficiency was | |
| | | the shell, made of | | on average 1.5 times | |
| | | alginate. Capsules | | higher in the capsules | |
| | | produced by two- | | cultured in the bioreactor | |
| | | fluidic electrostatic | | than the conventional bulk | |
| | | cospraying. | | Matrigel ® droplet static | |
| | | | | culture. | |
| Rotating | Retinal | Media | 50 mL vessel | For organoid | (DiStefano et |
| wall | organoids | supplemented with | (NASA- | manufacturing. | al., 2018) |
| bioreactor | (Murine | 2% v/v Matrigel ® | designed type) | Organoids at day 25 | |
| | PSC- | | operated at | presented similar | |
| | derived) | | gradually | maturation and | |
| | | | increased | transcriptome profile as | |
| | | | rotational | those at day 32 in static | |
| | | | speed from | culture. | |
| | | | 21.5 to 30 rpm | | |
| Customized | Brain | Cell aggregates | 12-well plate | For maintenance and | (Romero- |
| spinning | organoids | (embryoid bodies) | format with 3 | protocol optimization. | Morales et al., |
| bioreactor | (hPSC- | were embedded in | mL culture | Better nutrient exchange | 2019, Qian et |
| | derived) | Matrigel ®. After 7 | media per well | compared to static culture | al., 2018) |
| | | days, they were | which was | allowing for organoid sizes | |
| | | mechanically | changed every | of up to a few millimetres. | |
| | | dissociated from | 2 days. | Organoids cultured for | |
| | | Matrigel ® and | | more nearly 200 days. | |
| | | introduced in the | | Bioreactor commercially | |
| | | bioreactor. | | available (Spin∞ and | |
| | | | | SpinΩ). | |
| Microfluidic | Brain | Embryoid bodies | Microfluid chip | For maintenance, protocol | (Wang et al., |
| devices | organoids | were immobilized | culture: static | optimization and drug | 2018) |
| | (hiPSC- | into 40-50 μl of | for 6-8 hours | response assays. Perfused | |
| | derived) | Matrigel ® (300 | then | culture improved cortical | |
| | | aggregates/μL) per | continuous | development compared | |
| | | channel in the | perfusion at 25 | with static cultures. | |
| | | chip. | ml h⁻¹ for 5-22 | | |
| | | | days | | |

Accordingly, in a first aspect the present disclosure provides a method of expanding organoids, the method comprising: i) dissociating organoids into single cells and encapsulating the cells in gel beads; ii) culturing the encapsulated cells in an incubator under static conditions; iii) transferring the encapsulated cells to a fluidised bed bioreactor of a cell expansion apparatus, with a pump providing about 0.1 mm/s to about 5 mm/s superficial flow velocity in the bioreactor, to form organoids; and iv) retrieving the gel beads from the fluidised bed bioreactor for harvesting of the organoids.

In a second aspect, the present disclosure provides a cell expansion apparatus comprising: i) a fluidised bed bioreactor having a fluid inlet, a fluid outlet, a flow distributor proximal to the fluid inlet and a minimum volume of 50 ml; ii) a reservoir in fluid communication with a pump and a gas exchange unit; wherein the gas exchange unit is in fluid communication with the fluid inlet or the fluid outlet of the fluidised bed bioreactor, and wherein the fluid outlet of the fluidised bed bioreactor is in fluid communication with the reservoir.

In a further aspect the present disclosure provides a method of expanding organoids, the method comprising: i) dissociating organoids into single cells and encapsulating the cells in gel beads; ii) culturing the encapsulated cells in an incubator under static conditions; iii) transferring the encapsulated cells to a fluidised bed bioreactor, with a pump providing about 0.1 mm/s to about 5 mm/s superficial flow velocity in the bioreactor, to form organoids; and iv) retrieving the gel beads from the fluidised bed bioreactor for harvesting of the organoids, wherein the gel comprises about 0.1% to about 1% alginate, about 0.001% to about 0.02% gellan gum and about 2 mg/ml to about 10 mg/ml extracellular matrix protein.

The present inventors have determined that fluidised bed bioreactors can be used as part of a cell expansion apparatus to further scale up organoid culture, providing a more efficient and scalable expansion system. In a fluidised bed bioreactor, cell culture media enters from the bottom of the bioreactor and leaves from the top, suspending encapsulated organoid particles, and thereby allowing their entire surface to be in contact with the culture media. As a result, fluidised bed bioreactors are characterized by high rates of mass and heat transfer, low shear, and theoretically high cell density (thus smaller reactor volume for same final cell number), all of which are beneficial for growing organoids encapsulated within an appropriate matrix.

Fluidised bed bioreactors are advantageous for tissue engineering applications in general but have been largely overlooked in favour of stirred tanks and flasks, which are simpler culture systems. Fluidised bed bioreactors are well suited to particulate scaffolds, single cells or spheroids (Lucena-Thomas et al., 2020), and can provide a higher cell density and more defined culture environment than stirred tanks, as well as good mixing without the need of impellers, and thus less shear levels than stirred tanks. Fluidised bed bioreactors have been widely used in wastewater treatment applications (Bello et al., 2017) and increasingly explored as an artificial liver device (Legallais et al., 2000, Lu et al., 2016, Naghib et al., 2016, David et al., 2004), but not yet introduced in the organoid field.

The cell expansion apparatus of the disclosure is advantageous in that it can solve the problem of the need for a scalable bioreactor with good (non-rate limiting) mass transport conditions and a high culture density. Advantages of the apparatus include less material (e.g., culture media and other factors) being needed per unit number of organoids; more consistent time-averaged media supply/waste removal per organoid; ease of harvesting from bioreactor; the easy scalability of the apparatus and that it can be more space-efficient than other known systems, especially in terms of its footprint. Using a fluidised bed bioreactor can also be advantageous in that dead cells and/or any other debris which is present in the culture media can be “washed out” (elutriated) from the bioreactor chamber as culture medium passes through the bioreactor.

Other important advantages of fluidised bed bioreactors include the low shear stress imposed on cells due the absence of mechanical impellers (which can cause direct shock to cells) and foam formation (which can cause cell damage by microbubble bursting), which are common issues in stirred tank bioreactors. Moreover, by operating a bioreactor under fluidisation conditions as opposed to packed state, a higher liquid-solid interface area can be achieved when cells are suspended from one another by the liquid, and the consequent high degree of mixing significantly improves mass and heat transfer between media and cells, favouring the kinetics of cell metabolism.

### BRIEF DESCRIPTION OF THE DRAWINGS

The disclosure will now be described in detail, by way of example only, with reference to the figures.

FIG. 1 shows a schematic representation of a cell culture apparatus (FBB pilot).

FIG. 2 shows representative images of organoids cultured in a cell culture system of the present disclosure (A) and CXP1 (B). The organoids show similar morphology.

FIG. 3 shows an analysis of the organoid morphology, comparing organoids cultured in a cell culture system of the present disclosure (FBB pilot) and CXP1. Mean diameter (A), organoid density relative to matrix volume (B), organoid density to Matrigel® volume (C), viability (D) and cell increase (E). No significance observed. Error bars represented standard deviation (SD), N=3

FIG. 4 shows results from a drug response assay on organoids harvested from a cell culture system of the present disclosure (FBB Pilot) and CXP1. The results show the organoids behaved equivalently. Error bars represented standard error of the mean (SEM), N=1 and n=4 for CXP1 and n=12 (quadruplicate measurements of 3 vials) for CXP2 pilot.

FIG. 5 shows a schematic representation of a cell culture apparatus (FBB50)

FIG. 6 shows representative images of organoids cultured in a cell culture system of the present disclosure (FBB50) and CXP1. The organoids show similar morphology. Scale bar: 500 μm.

FIG. 7 shows representative images of organoids after being harvested from a cell culture system of the present disclosure (FBB50) and CXP1, frozen, thawed, replated into Matrigel® domes and cultured for 7 days. The organoid samples show similar morphology. Scale bar: 500 μm.

FIG. 8 shows MS3 counts and CellTiter-Glo® (CTG) assay results from the organoids after being harvested from a cell culture system of the present disclosure (FBB50) and CXP1, frozen, thawed, replated into Matrigel® domes and cultured for 7 days. The organoid samples show similar growth (MS3 counts) and metabolism/viability (CTG).

FIG. 9 shows results from a drug response assay on organoids harvested from a cell culture system of the present disclosure (FBB50) and CXP1. The results show the organoids behaved equivalently. Error bars represented standard error of the mean (SEM), N=1 and n=4.

FIG. 10 shows stiffness measurements from GAM (Matrigel®4.25-5 mg/mL protein, alginate 0.25% w/v, gellan gum 0.005% w/v), Matrigel® alone (7.67 mg/mL protein) and 1% w/v alginate using a using a uniaxial unconfined compression assay. These results show that GAM was demonstrated to be a soft matrix, with similar stiffness to Matrigel®. Alginate stiffness was significantly higher than GAM and Matrigel®.

FIG. 11 shows a schematic representation of a cell culture apparatus (FBB15 and FBB25).

FIG. 12 shows representative images of colorectal cancer and breast cancer organoids cultured in a cell culture system of the present disclosure (FBB25) and Matrigel® domes. The organoids show similar morphology. Scale bar: 200 μm.

### DETAILED DESCRIPTION

A cell expansion apparatus as described herein refers to an apparatus in which each of the described components can be independently modified, replaced or exchanged. This customizable apparatus provides particular advantages, especially in terms of scalability. Upon scale-up, process conditions commonly considered are mixing, mass/heat transfer and shear effects, all of which are highly improved using this system and are known to be beneficial for growing organoids. Moreover, this system offers a flexible operation in which cell feeding and waste removal can be automated and controlled based on the described sensor monitoring system for a more efficient process and reagent optimization, enabling higher densities to be achieved in a smaller footprint and reduced capital and operation costs.

The cell expansion apparatus is particularly suited to the expansion of organoids.

The term organoid simply means resembling an organ. Organoids are typically defined by three characteristics: self-organization, multicellularity and functionality (Lancaster and Knoblich, 2014b). Thus, the cells arrange themselves in vitro into the 3-dimensional (3D) organization that is characteristic for the organ in vivo, the resulting structure consists of multiple cell types found in that particular organ and the cells execute at least some of the functions that they normally carry out in that organ. For example, a prototypical organoid, the mouse intestinal organoid, grows as a single-layered epithelium organized into domains such that it resembles the in vivo intestinal crypt-villus architecture, comprising the different cell types of the intestine (enterocytes, goblet cells, Paneth cells, enteroendocrine cells and stem cells) and surrounding a cystic lumen (Sato et al., 2011)

In some embodiments the cells described herein are eukaryotic cells, and in some embodiments the cells are mammalian cells. Typically, cells are obtained from a multicellular organism, such as a human, and then cultured into organoids prior to further processing according to the methods described herein. The cells may be cancer cells, such as cells obtained from a tumor biopsy. The organoids may be grown from pluripotent stem cells (embryonic or induced) or from tissue biopsies containing adult stem cells. The cells may be healthy or representative of disease, such as those obtained from a heathy tissue or from a disease state, for example from a malignant tumor biopsy. When grown in vitro in conditions which support stem cell maintenance, organoids resemble the organ from which they were derived by recapitulating tissue specific cell types and 3D structure, genetic and physiologically relevant functions, while also being capable of continuous expansion.

The term expansion means to increase the total number of cells within a culture, either by increasing the total number of organoids within the culture or by increasing the number of cells per organoid or by a combination of the two. In contrast, “maintenance” of a culture (particularly a stem cell culture) refers to keeping the cells alive and viable (and for stem cells, keeping them in an undifferentiated “stem cell” state), but not necessarily increasing the total number of cells present. Any “excess” cells produced during maintenance are usually discarded to keep the total number of cells unchanged overall.

Fluidised bed bioreactor scaling has been traditionally done by hydrodynamic similarities based on dimensionless parameters, such as the simplified set proposed by Glicksman (Glicksman, 1984, Rüdisüli et al., 2012):

$$\frac{{u}_{0}}{{u}_{mf}} ;$$

$$\frac{{u}_{0}^{2}}{{gd}_{c}} ;$$

$$\frac{{h}_{c}}{{d}_{c}} ;$$

$$ϕ ;$$

$$psd$$

where u₀ is the given superficial velocity; u_{mf} the minimum fluidisation velocity; g the gravitational constant; d_{c} and h_{c} the diameter and height of the bioreactor, respectively; φ the sphericity of the particle; and psd the particle size distribution. These governing equations were initially created for gas-solid systems, but the same methodology has been extended to liquid-solid systems (Mendonca da Silva et al., 2020), due to similarities of design, geometry, hydrodynamic and kinetic properties.

The fluidised bed bioreactor of the cell expansion apparatus typically has a minimum volume of at least about 50 ml, or at least about 250 ml, or at least about 1000 ml. The maximum volume of the fluidised bed bioreactor may be about 10 L or about 9 L or about 8 L or about 7 L or about 6 L or about 5 L. In other words, the fluidised bed bioreactor may have a volume of from about 50 ml to about 10 L, or from about 250 ml to about 10 L, or from about 1000 ml to about 10 L.

Without being bound by theory, the present inventors have determined that the design of the fluidised bed bioreactor can be scaled while maintaining cell (e.g., organoid or spheroid) yield using rⁿ where r=radius of the column, and n=forces in the system i.e., inertia, viscosity, shear and gravity.

The fluid communication between the fluid outlet of the fluidised bed bioreactor and the reservoir typically comprises one or more sensors selected from an oxygen sensor, a pH sensor, a pressure sensor, a metabolite sensor, a temperature sensor or combinations thereof. Oxygen and/or metabolite sensors allow cell density over time to be indirectly monitored by analysing the cell culture media by metabolic data from in-line (e.g., oxygen) and off-line measurements (e.g., concentration of glucose, lactate, glutamine, ammonia, glutamate or combinations thereof).

Data from the one or more sensors can be continuously monitored and can be used to control operating parameters of the fluidised bed bioreactor such as flow rate, volume of culture media, gas exchange or combinations thereof.

Preferably, the apparatus includes at least one pressure sensor between the fluid outlet of the fluidised bed bioreactor and the reservoir, and/or at least one pressure sensor between the reservoir and the fluid inlet of the fluidised bed bioreactor comprises a pressure sensor. This allows the pressure of fluid flowing into the fluidised bed bioreactor to be monitored and/or the pressure of fluid flowing out of the fluidised bed bioreactor to be monitored.

Pressure sensors can thus be used to monitor cell density, because the size of the particles in the bioreactor (such as gel beads encapsulating cells or organoids) increases over time as the organoids grow. In particular, subtracting the pressure measured at or near the fluid outlet of the fluidised bed bioreactor from the pressure measured at or near the fluid inlet of the fluidised bed bioreactor provides the pressure drop across the fluidised bed bioreactor. Preferably, the cell expansion apparatus is operated to maintain a constant pressure drop across the fluidised bed bioreactor.

The metabolite sensor may be one or more lactate sensors. Lactate levels can provide a strong indication of how well the organoids are growing, especially when the organoids are cancer organoids.

After passing through the fluidised bed bioreactor cell culture media can be removed or returned to the reservoir, with or without recycling. Recycling of the cell culture media typically comprises passing the media through a series of steps to remove waste molecules of different sizes. The recycled cell culture media can then be returned to the reservoir. Alternatively, the after passing through the fluidised bed bioreactor the cell culture media can return to the reservoir without recycling.

The pump maintains fluidisation of the encapsulated organoids in the fluidised bed bioreactor, and ensures that nutrient and oxygen needs of cells being expanded in the apparatus can be met. Preferably the pump is a positive displacement pump. Such pumps have been found to successfully maintain fluidisation, meet the nutrient and oxygen needs of the cells and also ensure that cells are not washed out of the fluidised bed bioreactor with the flow of cell culture media. However, the pump also allows the flow rate of cell culture media to be increased as and when desired in order to elutriate the cells or gel beads containing encapsulated cells or organoids from the fluidised bed bioreactor.

The positive displacement pump may be a peristaltic pump. Such pumps typically comprise a flexible tube fitted inside a pump casing. A rotor inside the case has a number of ‘wipers’ or ‘rollers’ which compress the flexible tubing as they rotate by. The part of the tube under compression is closed, forcing fluid to move through the tube and as the tube opens to its natural state after the rollers pass, more fluid is drawn into the tube. Typically, there will be two or more rollers compressing the tube, trapping a body of fluid between them. The body of fluid is transported through the tube, toward the pump outlet. The flexible tube contains the fluid so that it does not directly contact the pump or pump components. This means that the fluid can be contained within sterile tubing, facilitating aseptic and sterile processes.

The cell expansion apparatus may further comprise an air pump connected to and in fluid communication with the gas exchange unit. Optionally, the fluid communication between the air pump and the gas exchange unit comprises a filter and/or an air flow meter.

The fluid communication between the gas exchange unit and the fluid inlet or the fluid outlet of the fluidised bed bioreactor may comprise one or more sensors selected from an oxygen sensor, a pH sensor, a pressure sensor, a metabolite sensor, temperature sensor or combinations thereof, the sensors being as described above. As discussed above, pressure, oxygen and/or metabolite sensors allow cell density to be indirectly monitored.

The fluidised bed bioreactor typically comprises a vertical column with a flow distributor at the bottom. Fluid enters the column from the bottom and leaves from the top. The upward flow of fluid suspends particles (e.g., gel beads encapsulating cells or organoids), so their entire surface is in contact with the fluid. The role of the distributor is to support the bed and to provide, as much as possible, a radial uniform liquid velocity distribution, thus minimising flow irregularities such as channeling and bulk circulation. The flow distributor therefore regulates the flow of fluid, e.g., culture media, into the fluidised bed bioreactors.

In some embodiments the flow distributor may be a porous substrate, such as a packed bed of solid particles or beads. The porosity of the substrate can be adjusted in accordance with, e.g., the size of the particles or beads, the size of the fluidised bed bioreactor and/or the type of cells being cultured. The particles or beads are preferably formed from a biocompatible and sterilisable inert material such as plastic or glass. For example, the flow distributor may comprise solid particles or beads with a diameter of about 0.5 mm to about 5 mm, or about 2 mm to about 4 mm. In some embodiments the flow distributor is a packed bed of glass beads, the beads having a diameter of about 3 mm.

The flow distributor may comprise single or multiple plates, each of which allows fluid to pass through the plate. Fluid flow is typically regulated by the size, density and pattern of holes or pores in the plates. The number and diameter of holes or pores in the plates should be balanced for good fluid distribution and pressure drop. If pressure drop is too low, the fluid will be poorly distributed, if too high, the system may leak or be unsafe. The plates are preferably formed from a biocompatible and sterilisable inert material such as plastic or glass.

In some embodiments the flow distributor may comprise a flow distributor plate and/or a flow distributor substrate. A flow distributor plate may be a plate comprising holes or pore through which fluid, such as culture media, can pass. The size, density and pattern of the holes or pores can be adjusted in accordance with, e.g., the size of the plate, the size of the fluidised bed bioreactor and/or the type of cells being cultured. Holes or pores in the distributor plate are preferably smaller than the particles (e.g., encapsulated organoids)) being cultured in the bioreactor. This can prevent the particles from becoming trapped within the holes or pores and blocking the distributor. For example, the flow distributor plate may comprise holes having a diameter of 3 mm or less, or 2 mm or less or 1 mm or less. Preferably, the flow distributor plate comprises holes having a diameter of about 1 mm.

A flow distributer substrate is preferably a porous substrate, such as sintered glass discs. The porosity of the substrate can be adjusted in accordance with, e.g., the size of the substrate, the size of the fluidised bed bioreactor and/or the type of cells being cultured. For example, the flow distributer substrate may comprise pores having a diameter of about 20 μm to about 500 μm, preferably about 60 μm to about 300 μm, more preferably about 100 to about 200 μm.

Preferably the flow distributor substrate is positioned between the fluid inlet of the fluidised bed bioreactor and the flow distributor plate.

Optionally the cell expansion system may include flow distributor structures, such as Pall Rings, within the fluidised bed bioreactor in order to prevent or reduce aggregation between gel beads. The flow distributor structures may be used in combination with or as an alternative to the flow distributor plates and/or substrates as described above.

The gas exchange unit can be particularly useful for oxygen-enrichment of the cell culture media passing through the cell expansion system. Suitable gas exchange units may comprise a silicon membrane and/or hollow fibre gas exchange unit.

Fluid communication within the expansion apparatus is typically provided by tubing through which culture medium can flow. The tubing may be flexible and can be formed from a biocompatible and sterilisable inert material such as silicone. The tubing is preferably transparent, which allows any bubbles or flow instabilities to be easily observed. Other suitable materials include polypropylene and polypropylene-based thermoplastic elastomers, such as PharMed®. Such materials can be well suited to withstanding the rigours of peristaltic pumping.

The minimum volume of the cell expansion apparatus, including the reservoir of cell culture media, fluidised bed bioreactor and tubing is typically at least about 250 ml, or at least about 1 L or at least about 5 L. In some embodiments, the maximum volume of the cell expansion apparatus, including the reservoir of cell culture media, fluidised bed bioreactor and tubing may be about 50 L or about 100 L, or about 150 L.

Preferably the components of the apparatus described herein are sterile and/or aseptic in order to avoid contamination of the cells being expanded in the apparatus.

The cell expansion apparatus is preferably a closed system. When the cell expansion apparatus is a closed system the entire apparatus is preferably sterile and/or aseptic. Indeed, the apparatus is advantageously easy to clean and reusable. The apparatus can also be easily sterilised between uses, e.g., by autoclaving.

The present disclosure also provides a method of expanding spheroids or organoids, the method comprising a method of expanding organoids, the method comprising: i) dissociating organoids into single cells and encapsulating the cells in gel beads; ii) culturing the encapsulated cells in an incubator under static conditions; iii) transferring the encapsulated cells to a fluidised bed bioreactor of a cell expansion apparatus, with a pump providing about 0.1 mm/s to about 5 mm/s superficial flow velocity in the bioreactor, to form organoids; and iv) retrieving the gel beads from the fluidised bed bioreactor for harvesting of the organoids.

In order that cells are under fluidisation and not carried away (elutriation) from the bioreactor chamber by the culture media, the liquid needs to be supplied within the minimum fluidisation velocity and the terminal velocity range. The pump may be operated to provide about 0.5 mm/s to about 4 mm/s superficial flow velocity in the bioreactor. Such a range has been found to be sufficient to maintain fluidisation, meet the nutrient and oxygen needs of the cells and also ensure that the gel beads are not washed out of the fluidised bed bioreactor with the flow of cell culture media. As discussed above, the flow velocity can be adjusted as and when desired in order to elutriate the gel beads.

The method may include (a) monitoring pressure of fluid flowing into the fluidised bed bioreactor, and/or (b) monitoring pressure of fluid flowing out of the fluidised bed bioreactor. When both (a) and (b) are monitored the pressure of (a) can be subtracted from the pressure of (b) to calculate the pressure drop. Preferably, the superficial flow velocity is adjusted to maintain a constant pressure drop.

Encapsulation of the gel beads can be performed, for example, using an electrospraying system, e.g., as described in Park et al (Int J Pharmaceut. 427 (2012) 417-425) and Wright et al (J. Biomed. Mater. Res. 102 (2013) 3393-3400). Alternatively, an extrusion dripping method can be used in which cells are suspended in gel solution and extruded into a gelation bath forming beads which can be collected and transferred to the fluidised bed bioreactor.

The encapsulated cells of step (ii) are typically cultured under static conditions for at least about 1 day.

The encapsulated cells of step (iii) can be cultured to form organoids of about 20 μm to about 700 μm in diameter. Organoids of greater than 700 μm in diameter have frequently been observed to develop necrotic cores, meaning that they cannot be used to accurately determine the effect of a toxicological compound since they are likely to have a pre-existing necrotic core which would give unrepresentative results. The encapsulated cells of step (iii) may be cultured to form organoids of about 20 μm to about 500 μm in diameter, preferably about 20 μm to about 200 μm in diameter, more preferably about 40 μm to about 90 μm in diameter.

The encapsulated cells of step (iii) may be cultured for about 3 to about 15 days, or about 4 to about 7 days. Typically, culture time can be determined by monitoring the size of the organoids, with a target diameter of about 40 μm to about 85 μm.

A culture time of ‘about X days’ as described herein typically refers to X days ±12 hours or X days±6 hours.

The cells may be encapsulated at approximately 200 cells/μL gel to approximately 2500 cells/μL gel. However, as the skilled person will be aware, the optimal seeding density will vary within this range depending on the cell or organoid line being expanded.

Preferably the gel is a hydrogel. Hydrogel as used herein refers to a system in which hydrophilic polymer chains are dispersed in an aqueous solution, such as an aqueous buffer solution or water. Typically, the hydrogel is in a gel state, such as a semi-solid state which retains shape. The aqueous buffer solution is preferably isotonic and/or pH neutral, both of which are beneficial for cell health. Suitable aqueous buffer solutions include phosphate buffered saline.

The hydrogel typically has a Young's modulus of less than 10 kPa when measured by uniaxial unconfined oscillatory compression. For example, the hydrogel may have a Young's modulus of about 5 kPa to about 10 kPa when measure by uniaxial unconfined oscillatory compression.

The gel may comprise about 0.2% to about 0.5% w/v alginate or about 0.2 to about 0.4% w/v alginate. In some embodiments the gel comprises about 0.2% to about 0.3% w/v alginate. The gel may comprise about 0.25% w/v alginate.

Alginate is a naturally occurring anionic polymer typically obtained from brown seaweed, which is known for its biocompatibility and ease of gelation. Alginate is known to be a whole family of linear copolymers containing blocks of (1,4)-linked β-D-mannuronate (M) and α-L-guluronate (G) residues. The blocks are composed of consecutive G residues, consecutive M residues, and alternating M and G residues. Alginates extracted from different sources differ in M and G contents as well as the length of each block. The alginate used in the compositions described herein can have a low M/G ratio (for example, G: approximately 65-70%; M: approximately 25-35%). G blocks have a higher affinity for calcium ions than M blocks. Thus, alginates with a higher M/G ratio are capable of creating more permeable, flexible and softer alginate gel matrices, whereas a lower M/G ratio leads to stronger structures.

Alginate forms gels in the presence of divalent and/or trivalent cations. The state of the alginate as described herein will be appropriate to the state in which is it being used. For example, when in a gel composition, such as a bead encapsulating organoids, the alginate will be in a gel state. Alternatively, when the alginate is being handled prior to gelation it is not in a gel state and will typically be in a sol state.

The gel may comprise about 0.002% to about 0.01% w/v gellan gum or about 0.003% to about 0.009% w/v gellan gum. In some embodiments the gel comprises about 0.004% to about 0.006% w/v gellan gum. The gel may comprise about 0.005% w/v gellan gum.

Gellan gum is an extracellular polysaccharide secreted by the microorganism Sphingomonas elodea (ATCC 31461) previously referred to as Pseudomonas elodea. It is available in two forms, high acyl typically containing 11-13% glyceryl groups and 4-5% acetyl groups, with the total amount of the acyl groups being in the range of 15-18% (weight percent) and low acyl typically containing less than 1% of glyceryl groups and less than 1% of acetyl groups, with the total amount of the acyl groups being below 2 wt %. The gellan gum used in the compositions described herein can be a low acyl gellan gum. Low acyl gellan gum can be prepared, for example, by the methods described in U.S. Pat. No. 8,609,377.

Gellan gum forms gels at low concentrations when hot solutions are cooled in the presence of gel-promoting cations. The state of the gellan gum as described herein will be appropriate to the state in which is it being used. For example, when in a gel composition, such as a bead encapsulating organoids, cells or spheroids, the gellan gum will be in a gel state. Alternatively, when the gellan gum is being handled prior to gelation it is not in a gel state and will typically be in a sol state.

The gel may additionally comprise a protein matrix, which provides extracellular matrix (ECM) proteins that can mimic the extracellular environment and act as a scaffold. The protein matrix typically comprises ECM proteins such as collagen I and/or collagen IV, as well as laminin. The protein matrix may additionally comprise ECM proteins such as entactin, perlecan or gelatin, or a combination thereof. The protein matrix may be an artificially sourced protein matrix or a biologically sourced protein matrix.

Biologically sourced protein matrices which may be used in the gel described herein are typically solubilised basement preparations extracted from the Engelbreth-Holm-Swarm (EHS) mouse sarcoma. EHS mouse sarcoma is a tumour rich in extracellular matrix (ECM) proteins such as laminin, collagen IV, heparan sulfate proteoglycans, entactin/nidogen, and a number of growth factors. Reduced growth factor biologically sourced protein matrices can also be used.

Suitable biologically sourced protein matrices for use in the gel compositions include protein matrices comprising laminin, entactin and collagen IV and optionally, heparin sulfate proteoglycan. Commercially available examples include Matrigel® (which comprises laminin, entactin and collagen IV), ECM Gel (which comprises laminin, collagen IV, entactin, and heparan sulfate proteoglycan), Cultrex® (which comprises laminin, entactin, collagen IV and heparin sulphate proteoglycan) and/or Geltrex™ (which comprises laminin, entactin, collagen IV and heparin sulphate proteoglycan). Preferably the biologically sourced protein matrix is Matrigel®.

Artificially sourced protein matrices may be PEG-based hydrogels, which are generally conjugated to key peptide residues from ECM proteins such as collagen and laminin. Polysaccharide products can also be used.

The gel may comprise an ECM protein content deriving from the protein matrix of about 2 mg/ml to about 10 mg/ml, or about 3 mg/ml to about 8 mg/ml. In some embodiments the gel comprises about 4 mg/ml to about 6 mg/ml ECM protein or about 4.25 mg/ml to about 5 mg/mL ECM protein. The gel therefore reduces the amount of protein matrix needed. The protein matrix component of the gel is therefore diluted down by the other components (i.e., the alginate and the gellan gum). Sufficient protein matrix is present to provide the biological cues needed by the cells or organoids, but problems associated with the variable nature of biologically sourced protein matrices are reduced to the dilution of this component. Dilution of the protein matrix component also allows the final protein concentration within the composition to be controlled.

The gel may further comprise organoids or cells. In some embodiments the gel comprises organoids.

The gel may therefore comprise about 0.2% to about 0.5% w/v alginate, about 0.002% to about 0.01% gellan gum, about 3 mg/ml to about 8 mg/ml ECM protein and organoids or cells. This blend of hydrogels typically has a Young's modulus of about 5 to about 10 kPa when measured by uniaxial unconfined oscillatory compression. In contrast, the present inventors have determined that 1% (w/v) alginate alone, i.e., when not used in combination with any other hydrogels, typically has a Young's modulus of about 36 kPa when measured by uniaxial unconfined oscillatory compression.

The gel beads may be about 200 μM to about 4000 μM in diameter. The gel beads may be about 1000 μm to about 3000 μm in diameter. For example, the gel beads might have a diameter of about 2.1 mm, or about 2.2 mm, or about 2.4 mm, or about 2.5 mm, or about 2.6 mm, or about 2.7 mm, or about 2.8 mm, or about 2.9 mm.

The gel beads of step (iv) can be retrieved by elutriation. In other words, the superficial flow velocity in the bioreactor can be increased to wash the beads out of the fluidised bed bioreactor. Alternatively, the gel beads can be aseptically retrieved from the fluidised bioreactor, for example by removing a top end piece of the bioreactor or by upending the bioreactor. When the flow of media through the bioreactor is stopped the gel beads will naturally settle to the bottom of the bioreactor from where they can be easily recovered, e.g., using an aspirator.

The gel beads can be dissolved in order to harvest the spheroids or organoids. Preferably the gel beads are dissolved using a dissolution buffer to breakdown the gel and release the organoids without damage. The dissolution buffer may comprise one or more solvents and can be optimised based on the hydrogel or hydrogels used to form the gel beads. For example, when the gel or gel blend comprises Matrigel® the dissolution buffer preferably comprises Cell Recovery Solution, which can remove the Matrigel® while avoiding dissociation of the cells of the organoids.

Optionally the retrieved organoids may be centrifuged to separate them from any remaining culture medium after the gel has been dissolved. Preferably the centrifuge settings are adjusted to increase the gravity and reduce the brake speed to prevent resuspension of the cell pellet.

Cell culture media are well known in the art and will be familiar to the skilled person. Typically, cell culture medium comprises amino acids, salts, glucose and vitamins and may also comprise iron and phenol red. A culture medium suitable for use in the cell expansion systems and methods described herein may be generated by modification of an existing cell culture medium. For example, the cell culture medium may be Dulbecco's modified Eagle medium (DMEM) and may comprise one or more additional components such as a nutrient mixture (e.g. Ham's F12), antibiotics/antifungals (e.g. penicillin/streptomycin), buffer (e.g. HEPES), glutamine, and n-Acetyl cysteine. The cell culture medium may additionally comprise a serum-free supplement, such as N2 Supplement and/or B27 Supplement.

### EXAMPLES

### Example 1

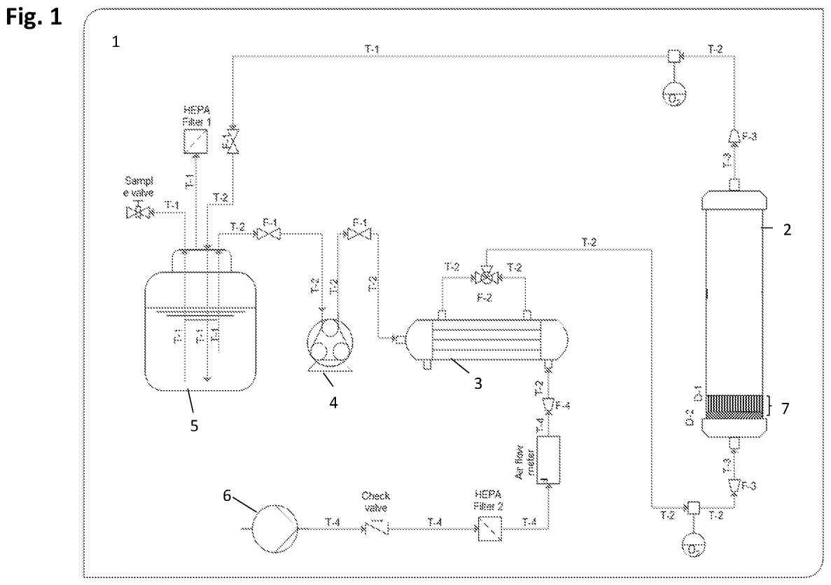

The pilot FBB expansion apparatus includes an incubator 1 containing a media reservoir 5, a fluidised bed bioreactor 2, which houses the encapsulated organoids and which contains a flow distributor 7, a peristaltic pump 4 for perfusion, a hollow fibre exchange module 3 and air pump 6 for gas-liquid mass transfer and oxygen sensors/detectors for measuring differential oxygen across the fluidised bed bioreactor 2, as shown in FIG. 1.

For the fluidised bed bioreactor, an Omnifit chromatography column (5 cm ID) was repurposed for organoid culture with threaded fixed ends (1 cm ID inlet and outlet). The flow distributor 7 set-up consisted of a sintered glass filter discs (5 cm OD×4 mm, 100-160 μm porosity) and a custom-made plastic distributor (1 mm diameter holes). The volume of the bed in the unexpanded state was between 30 to 60% of the reactor volume.

The operating conditions to fluidise the particles were determined to be between u_{mf}=0.66 mm/s and uₜ=4.22 mm/s. When operated at 0.94 mm/s, the fluidised bed bioreactor maintained good levels of oxygen concentration at the exit.

The process for culturing organoids using the cell culture system included the following steps:
1. Organoids were dissociated into single cells and encapsulated in hydrogel beads at 600 cell/μL.
2. Encapsulated organoids were maintained in the incubator for 2 days under static conditions, to allow cell recovery after organoid dissociation.
3. Encapsulated organoids were transferred to the fluidised bed bioreactor, operated at 0.94 mm/s fluid velocity for 3 days.
4. After the desired size of organoids was achieved (40-85 μm, for drug assays), the gel beads were retrieved from the fluidised bed bioreactor by stopping the pump, removing the top end piece of the fluidised bed bioreactor and aseptically removing the gel beads.
5. Organoids were harvested from the beads, characterized and frozen into cryovials for future use.

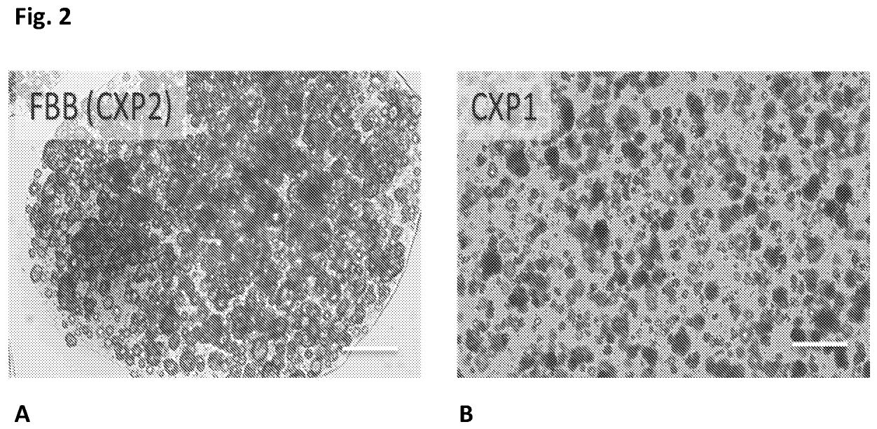

Organoids harvested from the cell culture system presented similar morphology to those cultured in CPX1 (as described in WO2018/011558) (FIG. 2).

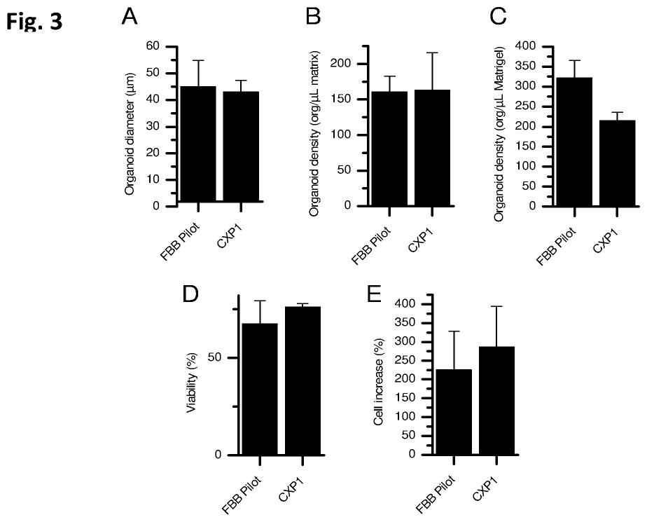

FIG. 3 shows that organoids harvested from the fluidised bed bioreactor and CXP1 presented similar diameter, final organoid densities (relative matrix volume or hydrogel volume), viability and cell percentage increase.

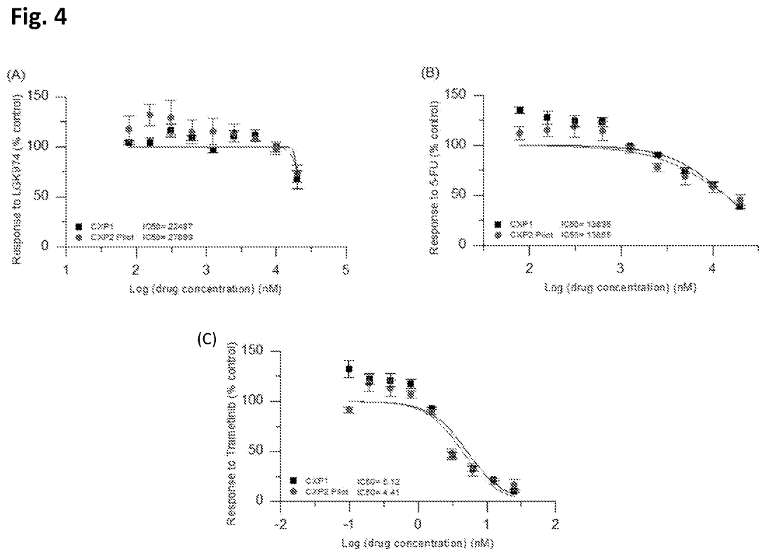

Once harvested from the fluidised bed bioreactor, organoids were replated into Matrigel® and showed an equivalent functional response for a panel of 3 drugs (FIG. 4).

The cell expansion system of the present disclosure therefore provides a scalable bioreactor with good (non-rate limiting) mass transport conditions and a high culture density. Other bioreactors that have been used to date, that use gels (e.g., Matrigel® or others) to culture organoids, do not have as high culture density nor the ability to elutriate the beads.

### Example 2

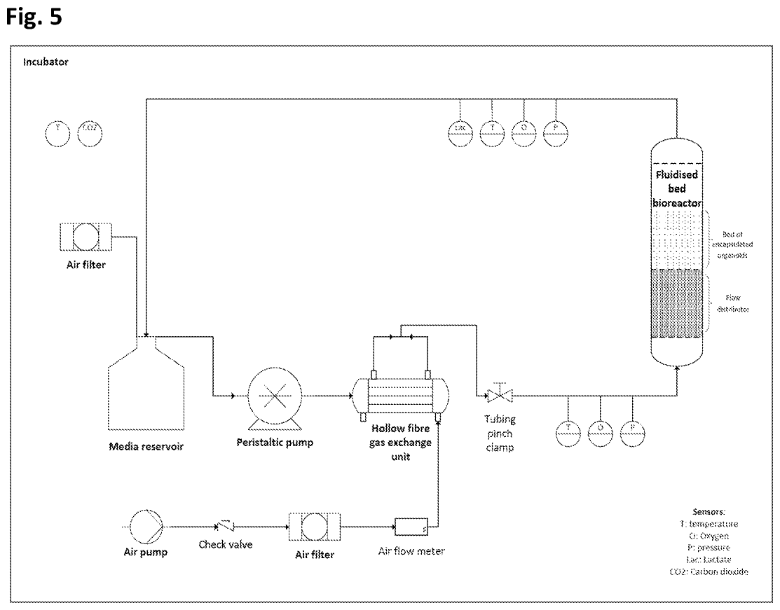

The FBB50 expansion apparatus includes an incubator containing a media reservoir, a peristaltic pump for perfusion, a hollow fibre exchange module and air pump for gas-liquid mass transfer, a fluidised bed bioreactor (50 mm ID), which contains a flow distributor (3 mm glass beads) and houses the encapsulated organoids, all of which are connected by tubing allowing communication between the components. The apparatus additionally includes temperature, oxygen and pressure sensors/detectors for measuring differential oxygen, temperature and pressure across the fluidised bed bioreactor, as shown in FIG. 5. A lactate sensor/detector is also included between the fluid outflow from the fluidised bed bioreactor and the media reservoir to monitor organoid growth and/or metabolism. Additional temperature and CO₂ sensors are positioned inside the incubator to monitor overall culture conditions inside the apparatus. Air filters are provided to the media reservoir and between the air pump and the hollow fibre gas exchange unit. An air flow meter is positioned between the air pump and the hollow fibre gas exchange unit. A pinch clamp is positioned on the tubing between the hollow fibre gas exchange unit and the fluid inlet to the fluidised bed bioreactor.

The process for culturing organoids using the cell culture system included the following steps:
1. Organoids were dissociated into single cells and encapsulated in hydrogel beads at 600 cell/μL.
2. Encapsulated organoids were maintained in the incubator for 2 days under static conditions, to allow cell recovery after organoid dissociation.
3. Encapsulated organoids were transferred to the fluidised bed bioreactor, operated at 0.94 mm/s fluid velocity for 3 days.
4. After the desired size of organoids was achieved (40-85 μm, for drug assays), which took 3 days, the gel beads were retrieved from the fluidised bed bioreactor by stopping the pump, removing the top end piece of the fluidised bed bioreactor and aseptically removing the gel beads.
5. Organoids were harvested from the beads, characterized and frozen into cryovials for future use.

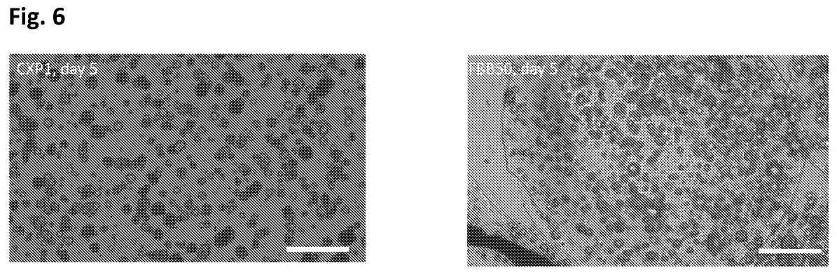

Organoids harvested from the FBB50 apparatus presented similar morphology to those cultured in CPX1 (as described in WO2018/011558) (FIG. 6).

The FBB50 apparatus produced almost 15× more organoids than a comparative process run with CPX1 and reduced the Matrigel® requirement by 40% (Table 1).

TABLE 1
FBB50 vs CXP1

| | | | Number of vials |
|---|---|---|---|
| | Organoids/μl | Organoids/μl | (at 130k |
| | Matrix | Matrigel ® | organoids/vial) |
| FBB50 | 121.27 | 208.30 | 72 vials |
| CXP1 | 115.55 | 126.76 | 5 vials |

The FBB50 apparatus additionally reduced the media volume requirements by 33% compared to CPX1 (Table 2).

TABLE 2
FBB50 vs CXP1

| | Total media volume | Media/Matrix volume |
|---|---|---|
| FBB50 | 800 | 10 |
| CXP1 | 89 | 15 |

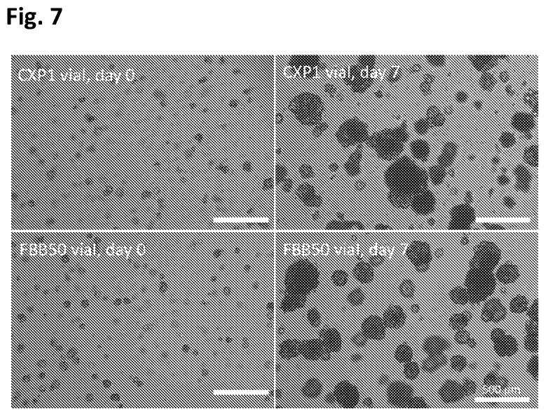

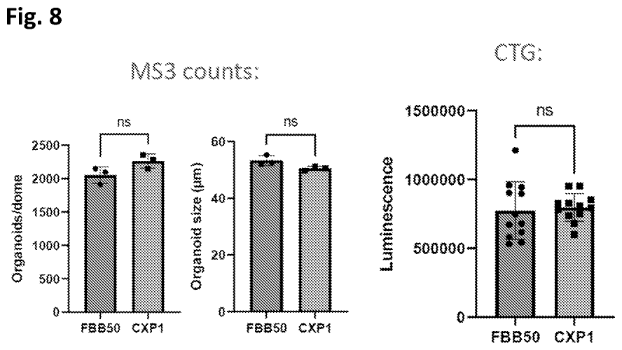

Organoids produced using the FBB50 apparatus and organoids produced using the CXP1 process were thawed and seeded into domes at 20 organoids/μl Matrigel®. After 7 days, end point MS3 counts (12×50 μl domes, 6-well plate) and CellTiterGlo assay (10 μl domes, 384 well plate) were performed. As shown in FIG. 7, once harvested from FBB50 and CXP1, frozen, thawed and replated into Matrigel®, organoid samples presented similar morphology and growth. As shown in FIG. 8, MS3 counts and CellTiterGlo assay results were similar for both groups of organoids.

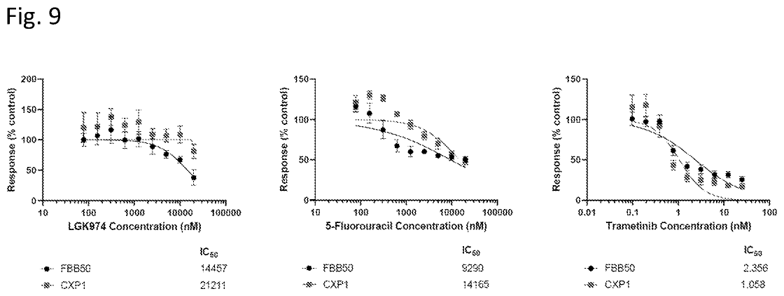

Organoids produced using the FBB50 apparatus and organoids produced using the CXP1 process were thawed and seeded into domes at 20 organoids/μl Matrigel®. After 7 days, end point drug assays (10 μl domes, 384 well plate) were performed. As shown in FIG. 9, once harvested from FBB50 and CXP1, frozen, thawed and replated into Matrigel®, organoids showed a similar response for a panel of three drugs.

The FFB cell expansion apparatus therefore provides significant advantages in terms of increased scalability, reduced capital and operating expenses, producing larger numbers of organoids, a smaller apparatus footprint, a more efficient process and reagent optimization.

### Example 3

A stiffness comparison was performed between GAM (Matrigel® 4.25 mg/mL protein, alginate 0.25% w/v, gellan gum 0.005% w/v), Matrigel® alone (7.67 mg/mL protein) and alginate alone (1% w/v) using a uniaxial unconfined compression assay.

In more detail, Matrigel®, alginate and GAM discs of 10 mm diameter and 2-2.6 mm thickness were prepared using the wells of 48-well cell culture plate as a mould. Similarly, Matrigel® gelation occurred at 37° C. and alginate and GAM gelation upon gently addition of 135 mM calcium chloride solution at 37° C. for 5 min, which was then removed by washing with culture media three times.

After 24 h at 37° C. in an incubator, allowed for the gels to set in culture media, the discs were gently removed from the well and the excess liquid dried with a tissue. Gel disc dimensions were measured using a calliper before loading between the parallel plates of the dynamic mechanical analyser (DMA1 from Mettler Toledo) equipment. The Young's modulus for each disc was determined by uniaxial unconfined oscillatory compression of 100 μm per second at 37° C. for 5 min.

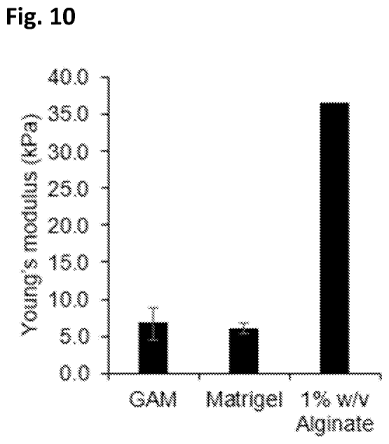

Results are shown in FIG. 10 and Table 3.

TABLE 3
Alginate, Matrigel ® and GAM stiffness comparison

| | | | Technical |
|---|---|---|---|
| | E* [kPa] | SD | replicates |
| GAM | 6.7 | 2.1 | n = 4 |
| Matrigel ® | 6.0 | 0.7 | n = 4 |
| 1% w/v | 36.4 | 0.0 | n = 1 |
| Alginate | | | |

The value for the 1% w/v alginate was consistent with the literature (38 and 25 kPa for G-type and M-type alginate (1.2% w/v) from Ceccaldi, 2012) using similar uniaxial compression assay.

Although pure alginate is well-established as a biocompatible material for cell encapsulation and tissue transplantation, for organoid culture, alginate does not contain necessary biologically active molecules (e.g., peptides or extracellular matrix proteins) to support stem cell maintenance and recapitulate supplementary biochemical, microstructural, and mechanical properties, such as, cell attachment, fate and migration.

GAM was demonstrated to be a soft matrix, with similar stiffness to Matrigel®, capable of degradation, while able to produce robust beads which were stable over time, which is not possible with pure Matrigel®.

### Example 4

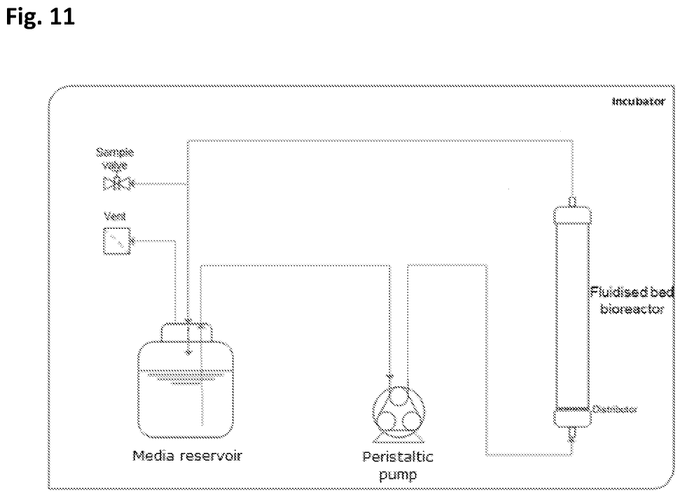

The FBB15 expansion apparatus includes an incubator containing a media reservoir, a fluidised bed bioreactor, which houses the encapsulated organoids, and which contains a flow distributor, and a peristaltic pump for perfusion, as shown in FIG. 11.

For the fluidised bed bioreactor, an Omnifit chromatography column (15 mm ID) was repurposed for organoid culture with threaded fixed ends (1 mm ID inlet and outlet). The flow distributor set-up consisted of polytetrafluoroethylene (PTFE) frit with 50 μm pores and a 10 μm Nylon membrane on top. The volume of the bed in the unexpanded state was between 30 to 60% of the reactor volume.

The operating conditions to fluidise the particles were determined to be between umf=0.66 mm/s and ut=4.22 mm/s. The following superficial velocities (u) were investigated: 0.71 mm/s, 0.94 mm/s and 1.89 mm/s, (umf<u<umf).

The process for culturing organoids using the cell culture system included the following steps:
1. Organoids were dissociated into single cells and encapsulated in hydrogel beads at 500 cell/μL, 600 cell/uL or 1000 cell/uL.
2. Encapsulated organoids were maintained in the incubator for 2 days under static conditions, to allow cell recovery after organoid dissociation.
3. Encapsulated organoids were transferred to the fluidised bed bioreactor, operated at 0.71 mm/s, 0.94 mm/s or 1.89 mm/s fluid velocity for 3 days.
4. After the desired size of organoids was achieved (40-85 μm, for drug assays), the gel beads were retrieved from the fluidised bed bioreactor by stopping the pump, removing the top end piece of the fluidised bed bioreactor and aseptically removing the gel beads.
5. Organoids were harvested from the beads, characterized and frozen into cryovials for future use.

As shown in tables A and B below the tested seeding densities and superficial velocities all resulted in organoids with a viability of >70%.

TABLE A
% viability was >70% at all superficial velocities

| Flow | Volume | Cell | |
|---|---|---|---|
| superficial | of | seeding | |
| velocity | beads | density | Viability |
| (mm · s) | (mL) | (cell/μL) | (%) |
| 0.71 | 6 | 600 | 97.9 |
| 0.94 | 6 | 600 | 76.1 |
| 1.89 | 6 | 600 | 90.8 |

TABLE B
% viability was >70% at all seeding densities

| Flow | Volume | Cell | |
|---|---|---|---|
| superficial | of | seeding | |
| velocity | beads | density | Viability |
| (mm · s) | (mL) | (cell/μL) | (%) |
| 0.94 | 6 | 500 | 84.6 |
| 0.94 | 6 | 1000 | 72.5 |
| 0.94 | 6 | 600 | 76.1 |

### Example 5

The FBB25 expansion apparatus includes an incubator containing a media reservoir, a fluidised bed bioreactor (25 mm ID), which houses the encapsulated organoids, and which contains a flow distributor (3 mm glass beads), and a peristaltic pump for perfusion, all of which are connected by tubing allowing communication between the components, as shown in FIG. 11.

The volume of the bed in the unexpanded state was between 30 to 60% of the reactor volume. The operating conditions to fluidise the particles were determined to be between umf=0.66 mm/s and ut=4.22 mm/s. When operated at 0.94 mm/s, the fluidised bed bioreactor maintained good levels of oxygen concentration at the exit.

The process for culturing organoids using the cell culture system included the following steps:
1. Colorectal cancer and breast cancer organoid lines were dissociated into single cells and encapsulated in hydrogel beads at 600 cells/uL and 1000 cells/uL matrix, respectively.
2. Encapsulated organoids were maintained in the incubator for 2 days under static conditions, to allow cell recovery after organoid dissociation.
3. Encapsulated organoids were transferred to the fluidised bed bioreactor, operated at 0.94 mm/s fluid velocity.
4. After 3 days (colorectal cancer organoids) and 13 days (breast cancer organoids), when the desired size of organoids was achieved (40-85 μm), the gel beads were retrieved from the fluidised bed bioreactor by stopping the pump, removing the top end piece of the fluidised bed bioreactor and aseptically removing the gel beads.
5. Organoids were harvested from the beads, characterized and frozen into cryovials for future use.

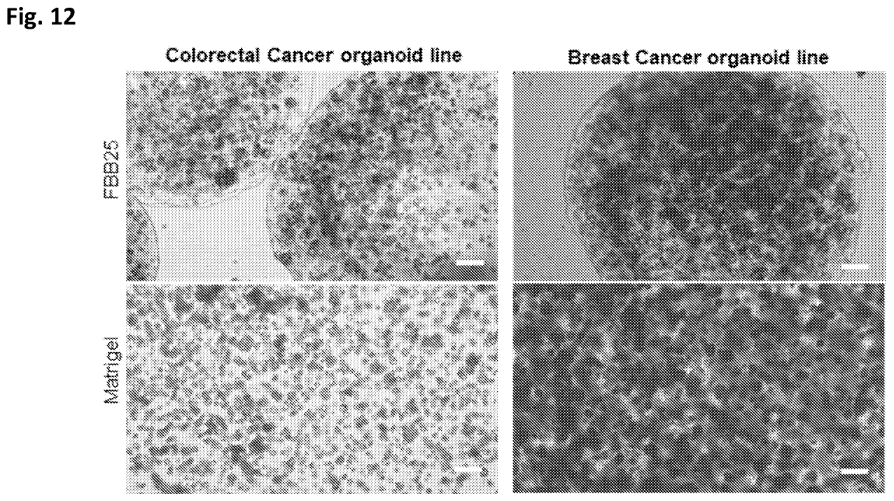

Organoids harvested from the cell culture system presented similar morphology to those cultured in Matrigel® domes (FIG. 12) for both organoid lines.

### REFERENCES

ASHOK, A., CHOUDHURY, D., FANG, Y. & HUNZIKER, W. 2020. Towards manufacturing of human organoids. Biotechnol Adv, 39, 107460.
BELLO, M. M., ABDUL RAMAN, A. A. & PURUSHOTHAMAN, M. 2017. Applications of fluidized bed reactors in wastewater treatment—A review of the major design and operational parameters. Journal of Cleaner Production, 141, 1492-1514.
BERGER, E., MAGLIARO, C., PACZIA, N., MONZEL, A. S., ANTONY, P., LINSTER, C. L., BOLOGNIN, S., AHLUWALIA, A. & SCHWAMBORN, J. C. 2018. Millifluidic culture improves human midbrain organoid vitality and differentiation. Lab Chip, 18, 3172-3183.
CECCALDI C, FULLANA SG, ALFARANO C, et al. Alginate Scaffolds for Mesenchymal Sten Cell Cardiac Therapy: Influence of Alginate Composition. Cell Transplantation. 2012; 21(9):1969-1984
CHOUDHURY, D., ASHOK, A. & NAING, M. W. 2020. Commercialization of Organoids. Trends Mol Med, 26, 245-249.
DAVID, B., DUFRESNE, M., NAGEL, M. D. & LEGALLAIS, C. 2004. In vitro assessment of encapsulated C3A hepatocytes functions in a fluidized bed bioreactor. Biotechnol Prog, 20, 1204-12.
DISTEFANO, T., CHEN, H. Y., PANEBIANCO, C., KAYA, K. D., BROOKS, M. J., GIESER, L., MORGAN, N. Y., POHIDA, T. & SWAROOP, A. 2018. Accelerated and Improved Differentiation of Retinal Organoids from Pluripotent Stem Cells in Rotating-Wall Vessel Bioreactors. Stem Cell Reports, 10, 300-313.
ELLIS, M. J., CHAUDHURI, J. & DALE, T. C. 2018. Methods for culturing organoids. WO2018011558A1.
FATEHULLAH, A., TAN, S. H. & BARKER, N. 2016. Organoids as an in vitro model of human development and disease. Nat Cell Biol, 18, 246-54.
GLICKSMAN, L. R. 1984. Scaling relationships for fluidized beds. Chemical Engineering Science, 39, 1373-1379.
LANCASTER, M. A. & KNOBLICH, J. A. 2014a. Generation of cerebral organoids from human pluripotent stem cells. Nat Protoc, 9, 2329-40.
LANCASTER, M. A. & KNOBLICH, J. A. 2014b. Organogenesis in a dish: modeling development and disease using organoid technologies. Science, 345, 1247125.
LANCASTER, M. A., RENNER, M., MARTIN, C. A., WENZEL, D., BICKNELL, L. S., HURLES, M. E., HOMFRAY, T., PENNINGER, J. M., JACKSON, A. P. & KNOBLICH, J. A. 2013. Cerebral organoids model human brain development and microcephaly. Nature, 501, 373-9.
LEGALLAIS, C., DORE, E. & PAULLIER, P. 2000. Design of a fluidized bed bioartificial liver. Artif Organs, 24, 519-25.
LU, J., ZHANG, X., LI, J., YU, L., CHEN, E., ZHU, D., ZHANG, Y. & LI, L. 2016. A New Fluidized Bed Bioreactor Based on Diversion-Type Microcapsule Suspension for Bioartificial Liver Systems. PLoS One, 11, e0147376.
LU, Y. C., FU, D. J., AN, D., CHIU, A., SCHWARTZ, R., NIKITIN, A. Y. & MA, M. 2017. Scalable Production and Cryostorage of Organoids Using Core-Shell Decoupled Hydrogel Capsules. Adv Biosyst, 1, 1-8.
LUCENA-THOMAS, J. P. D., BOONPRASIRT, P., LUETCHFORD, K., DE BANK, P. & ELLIS, M. 2020. Bed expansion properties of tissue engineering particles in a fluidised bed bioreactor. Biochemical Engineering Journal, 160.
MENDONCA DA SILVA, J., ERRO, E., AWAN, M., CHALMERS, S. A., FULLER, B. & SELDEN, C. 2020. Small-Scale Fluidized Bed Bioreactor for Long-Term Dynamic Culture of 3D Cell Constructs and in vitro Testing. Front Bioeng Biotechnol, 8, 895.
NAGHIB, S. D., PANDOLFI, V., PEREIRA, U., GIRIMONTE, R., CURCIO, E., DI MAIO, F. P., LEGALLAIS, C. & RENZO, A. D. 2016. Expansion properties of alginate beads as cell carrier in the fluidized bed bio artificial liver. Powder Technology, 300.
NOVOSEL, E. C., KLEINHANS, C. & KLUGER, P. J. 2011. Vascularization is the key challenge in tissue engineering. Advanced Drug Delivery Reviews, 63, 300-311.
OVANDO-ROCHE, P., WEST, E. L., BRANCH, M. J., SAMPSON, R. D., FERNANDO, M., MUNRO, P., GEORGIADIS, A., RIZZI, M., KLOC, M., NAEEM, A., RIBEIRO, J., SMITH, A. J., GONZALEZ-CORDERO, A. & ALI, R. R. 2018. Use of bioreactors for culturing human retinal organoids improves photoreceptor yields. Stem Cell Res Ther, 9, 156.
PHELAN, M. A., LELKES, P. I. & SWAROOP, A. 2018. Mini and customized low-cost bioreactors for optimized high-throughput generation of tissue organoids. Stem Cell Investig, 5, 33.
PRZEPIORSKI, A., SANDER, V., TRAN, T., HOLLYWOOD, J. A., SORRENSON, B., SHIH, J. H., WOLVETANG, E. J., MCMAHON, A. P., HOLM, T. M. & DAVIDSON, A. J. 2018. A Simple Bioreactor-Based Method to Generate Kidney Organoids from Pluripotent Stem Cells. Stem Cell Reports, 11, 470-484.
QIAN, X., JACOB, F., SONG, M. M., NGUYEN, H. N., SONG, H. & MING, G. L. 2018. Generation of human brain region-specific organoids using a miniaturized spinning bioreactor. Nat Protoc, 13, 565-580.
ROMERO-MORALES, A. I., O'GRADY, B. J., BALOTIN, K. M., BELLAN, L. M., LIPPMANN, E. S. & GAMA, V. 2019. Spinoo: an updated miniaturized spinning bioreactor design for the generation of human cerebral organoids from pluripotent stem cells. HardwareX, 6, e00084.
RÜDISÜLI, M., SCHILDHAUER, T. J., BIOLLAZ, S. M. A. & VAN OMMEN, J. R. 2012. Scale-up of bubbling fluidized bed reactors—A review. Powder Technology, 217, 21-38.
SATO, T., STANGE, D. E., FERRANTE, M., VRIES, R. G., VAN E S, J. H., VAN DEN BRINK, S., VAN HOUDT, W. J., PRONK, A., VAN GORP, J., SIERSEMA, P. D. & CLEVERS, H. 2011. Long-term expansion of epithelial organoids from human colon, adenoma, adenocarcinoma, and Barrett's epithelium. Gastroenterology, 141, 1762-72.
SCHNEEBERGER, K., SANCHEZ-ROMERO, N., YE, S., VAN STEENBEEK, F. G., OOSTERHOFF, L. A., PLA PALACIN, I., CHEN, C., VAN WOLFEREN, M. E., VAN TIENDEREN, G., LIESHOUT, R., COLEMONTS-VRONINKS, H., SCHENE, I., HOEKSTRA, R., VERSTEGEN, M. M. A., VAN DER LAAN, L. J. W., PENNING, L. C., FUCHS, S. A., CLEVERS, H., DE KOCK, J., BAPTISTA, P. M. & SPEE, B. 2019. Large-Scale Production of LGR5-Positive Bipotential Human Liver Stem Cells. Hepatology.
WANG, Y., WANG, L., GUO, Y., ZHU, Y. & QIN, J. 2018. Engineering stem cell-derived 3D brain organoids in a perfusable organ-on-a-chip system. RSC Advances, 8, 1677-1685.

1. A method of expanding organoids, the method comprising:
i) dissociating organoids into single cells and encapsulating the cells in gel beads;
ii) culturing the encapsulated cells in an incubator under static conditions;
iii) transferring the encapsulated cells to a fluidised bed bioreactor of a cell expansion apparatus, with a pump providing about 0.1 mm/s to about 5 mm/s superficial flow velocity in the bioreactor, to form organoids; and
iv) retrieving the gel beads from the fluidised bed bioreactor for harvesting of the organoids.

2. The method of claim 1, wherein the gel is a hydrogel.

3. The method of claim 2, wherein the hydrogel has a Young's modulus of less than 10 kPa.

4. The method of claim 1, wherein the gel comprises about 0.1% to about 1% alginate, about 0.001% to about 0.02% gellan gum and about 2 mg/ml to about 10 mg/ml extracellular matrix protein.

5. The method of claim 1, wherein the pump is operated to provide about 0.5 mm/s to about 4 mm/s superficial flow velocity.

6. The method of claim 1, further comprising (a) monitoring pressure of fluid flowing into the fluidised bed bioreactor, and (b) monitoring pressure of fluid flowing out of the fluidised bed bioreactor.

7. The method of claim 6, further comprising subtracting the pressure of (a) from the pressure of (b) to calculate the pressure drop, and (c) adjusting the superficial flow velocity to maintain a constant pressure drop.

8. The method of claim 1, wherein the gel beads are about 200 μM to about 4000 μM in diameter.

9. The method of claim 1, wherein:
the encapsulated cells of step (ii) are cultured under static conditions for at least about 1 day; and/or
the encapsulated cells of step (iii) are cultured to form organoids of about 20 μm to about 700 μm; and/or
the encapsulated cells of step (iii) are cultured for about 3 to about 15 days.

10-11. (canceled)

12. The method of claim 1, wherein the cells are encapsulated at approximately 200 cells/μL gel to approximately 2500 cells/μL gel.

13. A cell expansion apparatus comprising:
i) a fluidised bed bioreactor having a fluid inlet, a fluid outlet, a flow distributor proximal to the fluid inlet and a minimum volume of 50 ml; and
ii) a reservoir in fluid communication with a pump and a gas exchange unit;
wherein the gas exchange unit is in fluid communication with the fluid inlet or the fluid outlet of the fluidised bed bioreactor, and
wherein the fluid outlet of the fluidised bed bioreactor is in fluid communication with the reservoir.

14. The cell expansion apparatus of claim 14, wherein the fluid communication between the fluid outlet of the fluidised bed bioreactor and the reservoir comprises one or more sensors selected from the group consisting of an oxygen sensor, a pH sensor, a pressure sensor, a metabolite sensor, and combinations thereof.

15. The cell expansion apparatus of claim 14, wherein the fluid communication between the fluid outlet of the fluidised bed bioreactor and the reservoir comprises a pressure sensor.

16. The cell expansion apparatus of claim 13, wherein the pump is a positive displacement pump.

17. The cell expansion apparatus of claim 16, wherein the positive displacement pump is a peristaltic pump.

18. The cell expansion apparatus of claim 13, wherein the system further comprises an air pump connected to and in fluid communication with the gas exchange unit, and wherein the fluid communication between the air pump and the gas exchange unit comprises a filter and/or an air flow meter.

19. (canceled)

20. The cell expansion apparatus of claim 13, wherein the fluid communication between the gas exchange unit and the fluid inlet or the fluid outlet of the fluidised bed bioreactor comprises one or more sensors selected from the group consisting of an oxygen sensor, a pH sensor, a pressure sensor, a metabolite sensor, and combinations thereof.

21. The cell expansion apparatus of claim 13, wherein the fluid communication between the reservoir and the fluid inlet of the fluidised bed bioreactor comprises a pressure sensor.

22-23. (canceled)

24. The cell expansion apparatus of claim 13, wherein the gas exchange unit is a silicon membrane and/or hollow fibre gas exchange unit.

25. (canceled)

26. The method of claim 1, wherein the gel comprises about 0.2% to about 0.5% w/v alginate, about 0.002% to about 0.01% gellan gum, and about 3 mg/ml to about 8 mg/ml extracellular matrix protein.
